(19) United States
(12) Patent Application Publication
(10) Pub. No.: US 2022/0137403 A1
(43) Pub. Date: May 5, 2022

(54) METHOD AND ARRANGEMENT FOR CLEANING A SENSOR

(71) Applicant: KEMIRA OYJ, Helsinki (FI)

(72) Inventors: Greg LAND, Atlanta, GA (US); David SOPER, Atlanta, GA (US); Marco STAMMEGNA, Atlanta, GA (US)

(21) Appl. No.: 17/430,946

(22) PCT Filed: Feb. 17, 2020
(86) PCT No.: PCT/FI2020/050098

§ 371 Date: Aug. 13, 2021

Related U.S. Application Data
(60) Provisional application No. 62/805,979, filed on Feb. 15, 2019.

(30) Foreign Application Priority Data
Mar. 14, 2019 (FI) 20195186

(51) Int. Cl. G02B 27/00; G01N 33/18; C02F 1/00

(52) U.S. Cl. CPC G02B 27/0006; G01N 33/1806; G01N 33/1813; C02F 2209/11; C02F 2209/05; C02F 2209/06; C02F 2209/08; C02F 1/001

(57) ABSTRACT

The invention relates to a method for cleaning a sensor in a wastewater monitoring arrangement comprising at least one sensor with a sensor surface, such as an optical sensor with a window, lens, or the like. In the monitoring arrangement, during a normal operation mode, a sample flow of wastewater is arranged to flow past the sensor surface and the sensor is arranged to provide measurement values that describe a quality parameter of the wastewater. The method comprising steps of: starting a sensor cleaning cycle by discontinuing the sample flow; starting a cleaning liquid flow; arranging the cleaning liquid flow towards the sensor surface; mechanically cleaning the sensor surface by an automatic cleaning device; discontinuing the cleaning liquid flow after a predetermined cleaning time and ending the cleaning cycle; and starting the wastewater flow again.

### CROSS-REFERENCES TO RELATED APPLICATIONS

This application is a national phase application of international application number PCT/FI1010/050098 filed on Feb. 17, 2020 claiming priority from U.S. provisional application No. 62/805,979 filed on Feb. 15, 2019 and from Finnish application FI20195186 filed on Mar. 14, 2019, all of which are incorporated herein by reference.

### FIELD OF INVENTION

The present invention relates to a method and arrangement for cleaning a sensor in a wastewater monitoring according to the preambles of the enclosed independent claims.

### BACKGROUND

Environmental regulations require effective wastewater treatment in various processes in industry, agriculture, municipal applications and the like. Wastewater treatment can be performed by using chemical, biological or physical means, or their combinations. Proper chemical treatment regime requires information about the quality of the wastewater. Also, the effectiveness of the performed wastewater treatment can be controlled by monitoring one or more parameters of the treated wastewater. The monitoring, both before, during and after the wastewater treatment can be done by using sensors, for example optical sensors, that measure the desired parameter from the wastewater and transfer the measurement information to the monitoring system which then adjust various process parameters, e.g. chemical dosage, according to the obtained measurement values.

Wastewater can be a demanding environment for the sensors. Wastewater often contain contaminants, such as foreign solid matter (e.g. rags, particular material), fats, oils, grease, which lead a quick deposit build-up on the sensor surface. For example, lenses or windows of optical sensors become covered at least partially by biofilm, which leads to occlusions. Foreign solid matter or dirt may also become attached to the sensor surface. Deposits on sensor surface may lead to erroneous measurement readings and/or drift in measurement readings. In order to combat or avoid these problems, sensor surfaces should be kept clean. The deposit problem is especially pronounced when a sensor is used to measure the characteristics of the wastewater prior to biological or chemical treatment.

Conventionally efficient cleaning of sensor surfaces is mostly performed manually. Sensor and sensor system manufacturers try to extend the manual cleaning interval by utilizing various factory installed cleaning devices. In some applications these cleaning devices may provide adequate results when used for sensors, which monitor the treated wastewater, i.e. discharge from the wastewater treatment process. However, the present cleaning devices are seldom capable of providing acceptable cleaning results for sensors monitoring the receiving waters of the wastewater treatment system, i.e. non-treated incoming water. These sensors for receiving waters may require daily manual cleaning due to the fast fouling and deposit build-up on the sensor surface. The frequency of the manual sensor cleaning requires additional amount of work and may cause occupational health risks. On the other hand, in case the sensor surfaces are not regularly cleaned, the monitoring system controlling, for example, chemical dosages to the treatment system does not receive reliable measurement data. This may lead to incorrect dosage and make the chemical dosage based on real-time wastewater properties nearly impossible. Consequently, there is a need for efficient solutions for cleaning sensor surfaces, which are in contact with wastewater, especially with receiving wastewater.

### SUMMARY OF THE INVENTION

The object of the present invention is to minimize or even eliminate the disadvantages existing in the prior art.

One object of the present invention is to provide a method and an arrangement for efficient cleaning of a sensor surface in a wastewater monitoring.

A further object of the present invention is to provide easy and simple way of reducing fouling of and/or deposit build-up on a sensor surface in contact with wastewater, especially with receiving wastewater.

All the described embodiments and advantages apply both for the method and the arrangement according to the present invention, when applicable, even if not always explicitly stated so.

These objects are achieved by the features disclosed in the independent claim and the invention is defined by the features of the enclosed independent claim. Some preferred embodiments of the present invention are presented in the dependent claims.

Typical method according to the present invention for cleaning a sensor in a wastewater monitoring arrangement comprising at least one sensor with a sensor surface, such as an optical sensor with a window, lens, or the like, in which monitoring arrangement, during a normal operation mode, a sample flow of wastewater is arranged to flow past the sensor surface and the sensor is arranged to provide measurement values which describe a quality parameter of the wastewater, the method comprising steps of:
starting a sensor cleaning cycle by discontinuing the sample flow,
starting a cleaning liquid flow,
arranging the cleaning liquid flow towards the sensor surface,
mechanically cleaning the sensor surface by an automatic cleaning device,
discontinuing the cleaning liquid flow after a predetermined cleaning time and ending the cleaning cycle, and
starting the wastewater flow again.

Typical arrangement according to the present invention for a wastewater monitoring comprises at least one sensor with a sensor surface, such as an optical sensor with a window, lens, or the like, in which arrangement during a normal operation mode a sample flow of wastewater is arranged to flow past the sensor surface and the sensor is arranged to provide measurement values which describe a quality parameter of the wastewater, the arrangement comprising
a first reservoir for cleaning liquid, connections for leading the cleaning liquid from the first reservoir to the sensor and against the sensor surface, and transfer means for transferring the cleaning liquid from the reservoir to the sensor,
optionally means for heating the cleaning liquid to an elevated temperature,
automatic cleaning device for mechanically cleaning the sensor surface,
a control unit, which is in functional contact with at least the transfer means and the automatic cleaning device, and which is arranged to carry out a cleaning cycle according to the method of the present invention.

Now it has been surprisingly found that a use of cleaning liquid together with mechanical cleaning with an automatic cleaning device gives surprising improvements in the cleaning results of a sensor surface. It has been observed that the use of the arrangement and the method according to the present invention may prolong the manual cleaning interval from <48 hours up to, and even over, 2 weeks, preferably 4 weeks, sometimes up to 6 weeks. The sensor cleaning cycle may be performed fully automatically, at predetermined time intervals or when desired or deemed necessary. Automatized operation makes the regular cleaning of the sensor surface easy and fast and it does not require additional amount of work. It is highly unexpected that the automatized use of cleaning liquid and mechanical cleaning can produce such a significant reduction in required manual cleaning labour. The arrangement according to the invention could be considered a self-cleaning sensor station or self-cleaning sensor arrangement.

The present invention is suitable for wastewater monitoring arrangements that comprise at least one sensor with a sensor surface. The monitoring arrangement may comprise a plurality of sensors, such as two, three or more sensors, which preferably are different from each other and measure different parameters. The sensor may be an optical sensor, such as spectrophotometric sensor; electrochemical sensor, such as pH electrode or ion-selective electrode; biosensor; or a microwave sensor. According to one preferable embodiment the sensor is an optical sensor with a sensor surface, such as lens, window, fibre optics or the like, that is to be cleaned. The sensor may provide direct or indirect measurement data for the wastewater monitoring on turbidity, conductivity, pH, chemical oxygen demand (COD), biological oxygen demand (BOD), total dissolved gas (TDG), content of specific heavy metals, content of various species of nitrogen, sulfur and/or phosphorous species. One sensor may provide measurement data on several parameters. The arrangement of the present invention can be used to clean the sensor surface of any of the above-mentioned sensors.

According to one preferable embodiment the arrangement comprises at least one optical sensor that provides measurement data on sulfide content of the wastewater. Preferably, the arrangement may further comprise a pH measuring sensor, wherein the pH of the sample flow may be measured.

In case the arrangement comprises two or more sensors, each sensor may have its own automatic cleaning device arranged for the mechanical cleaning of the sensor surface. Alternatively, the arrangement may comprise cleaning devices arranged in the vicinity of only those sensor surfaces which are especially susceptible for measurement errors in case of fouling of or deposit build-up on the sensor surface. More robust sensors in the monitoring arrangement may be efficiently cleaned solely with the contact of the cleaning liquid and optional chemical cleaning agent.

The present invention is especially suitable for wastewater monitoring which use at least one sensor to monitor the characteristics or quality of the receiving waters to the wastewater treatment. The receiving waters may contain for example, flushable wipes, feminine hygiene products, fat oils and grease from restaurants. The receiving waters may originate or comprise also local industrial wastewaters from breweries, agriculture, livestock farming, factories, etc. According to one embodiment the receiving waters may be municipal wastewater, food industry wastewater, agricultural wastewater, livestock farming wastewater, paper, board or pulp mill raw water, or metallurgical industry water, such as waters from zinc smelting facilities. The present invention is suitable for all wastewater monitoring which are required to operate under harsh environmental conditions.

The arrangement comprises a control unit, which is in functional contact with the necessary transfer means, such as pumps and actuated valves, of the arrangement as well as the mechanical cleaning device. The control unit is arranged to carry out the cleaning cycle in a desired manner. The control unit comprises a Programmable Logic Controller (PLC) and usually a user interface, e.g. touch screen, which can be used for control of the operation of the arrangement, for example for setting up or selecting the cleaning cycle intervals, used chemical cleaning agent(s) and/or cleaning cycle sequence.

During the normal operation mode, a sample flow of wastewater is arranged to flow past the sensor surface(s). The sample flow may be a by-pass flow taken from a main wastewater flow. The arrangement may comprise sample transfer means, such as a sample inlet valve, a sample discharge valve, and/or a sample pump for transfer of the sample flow from the main wastewater flow and past the sensor surface. The sensor is arranged to provide measurement values, i.e. data, to a central control unit of the wastewater treatment system on the selected quality parameter of the wastewater. The measurement values are transferred from the sensor to the control unit of the wastewater treatment system, where the obtained measurement values are used to monitor or control the wastewater quality and, optionally, to adjust a feed of one or more treatment chemicals to the wastewater treatment system on basis of the measurement values.

The sensor cleaning cycle may be started or activated regularly at predetermined time intervals, for example once an hour or once in two or three hours. Alternatively, or in addition, the sensor cleaning cycle may be started if the obtained measurement values have steadily drifted or diverged over a predetermined threshold level. The control unit may contain preprogrammed threshold level values or make trend analysis of the obtained measurement values from the end of preceding cleaning cycle. Typically, the time interval between two successive sensor cleaning cycles may be 0.5-3 h, preferably 1-1.5 h. The suitable time interval may be selected as needed, depending on the process application and/or wastewater type or quality.

The cleaning cycle is started by discontinuing the sample flow past the sensor surface, for example by closing the sample discharge valve and discontinuing sample flow transfer. The sample inlet valve is usually kept open at this stage. The control unit is in functional contact with the sample transfer means, such as the sample pump, the sample discharge valve and the sample inlet valve, and provides necessary control commands. The cleaning liquid flow is started, and the cleaning liquid is arranged to flow from a first reservoir towards the sensor surface. Usually this means that the flow direction of the cleaning liquid flow is opposite to the flow direction of the sample flow during normal operation mode. According to one preferable embodiment the cleaning liquid flow may be arranged towards the sensor surface at a straight angle. This means that the cleaning liquid flow is arranged to contact the sensor surface directly which ensures the efficient removal of deposits, such as biofilm and/or other possible build-up attached to the sensor surface. The control unit is in functional contact with the transfer means of the cleaning liquid, such as the cleaning liquid pump.

The cleaning liquid flow is allowed to flow towards the sensor surface for a predetermined pre-wash time, typically <1 min, more typically 0.25-1 min. In this manner the sensor surface is rinsed, and the breakdown of the deposits, biofilm or build-up is started. The pre-wash time may be adjusted, if desired, via the user interface of the control unit.

The cleaning liquid may be an aqueous liquid, preferably water. According to one preferable embodiment of the invention the cleaning liquid is heated to an elevated temperature and the arrangement comprises means for heating the cleaning liquid to desired elevated temperature before it is led towards the sensor surface. The cleaning liquid may be heated to or have an elevated temperature of at least 50° C., preferably at least 70° C., more preferably at least 75° C. The cleaning liquid temperature may preferably be in the range of 70-95° C., more preferably 75-85° C. The elevated temperature of the cleaning liquid improves the removal of grease, fats and oils from the sensor surface.

After the pre-wash with the cleaning liquid the sensor surface is mechanically cleaned by an automatic cleaning device. The automatic cleaning device or its part is brought into contact with the sensor surface, usually by reciprocating motion, whereby the deposits on the sensor surface are detached and removed. The automatic cleaning device may be operated by any suitable operating means, for example by a pneumatic cylinder. The operating means receive necessary operational commands from the control unit.

According to one embodiment the automatic cleaning device have in its first end a cleaning head, which comes into contact with the sensor surface. The cleaning head may be in form of a brush or a like, or the cleaning head may be in form of a wiper or spatula, possibly provided with protrusions. Protrusions may be prepared from another material than the spatula or wiper body.

After the mechanical cleaning of the sensor surface the cleaning liquid flow may started again and allowed to flow a predetermined rinse time, usually <1 min, typically 0.25-0.75 min. The rinse time is usually shorter than the pre-wash time preceding the mechanical cleaning. The cleaning liquid flow transfers the deposits detached by mechanical cleaning away from the sensor surface.

In the end of the cleaning cycle the cleaning liquid flow is discontinued, the cleaning liquid valve is closed, and the sample discharge valve is opened, whereafter the wastewater flow past the sensor surface is started again.

According to one preferable embodiment the cleaning liquid flow may comprise at least one a chemical cleaning agent, such as a detergent, at least during a part of the cleaning cycle. The chemical cleaning agent may be selected from weak acids, weak bases, chelating agents, detergents, surfactants or any suitable mixtures thereof. Preferably the chemical cleaning agent may comprise a weak acid and/or a detergent for removal of oil, grease or fat from the sensor surface. The arrangement may comprise one or several second reservoirs for the chemical cleaning agent(s), as well as connections for leading the chemical cleaning agent(s) to the sensor surface. A second transfer means, such as pump, or plurality of second transfer means may be used for transferring the chemical cleaning agent(s) from the second reservoir(s) to the sensor surface.

The chemical cleaning agent may be fed into the cleaning liquid flow, either continuously or periodically. Alternatively, the cleaning liquid flow and the chemical cleaning agent may be fed to the sensor surface sequentially. According to one preferable embodiment the cleaning liquid is first arranged to flow towards the sensor surface for a predetermined pre-wash time. After that at least one chemical cleaning agent is introduced into the cleaning liquid flow. The combined flow of cleaning liquid and chemical cleaning agent is allowed to flow towards a predetermined washing time. After that the sensor is mechanically cleaned and finally rinsed with cleaning liquid, as described above.

The arrangement may further comprise a filter, which is arranged before the sensor in a flow direction of the sample flow during the normal operation mode, for removal solid and particulate material from the sample flow. In this manner the transfer of solid material, such as rags, fibers, particles and the like, to the sensor surface may be reduced. The filter may be any suitable filter.

According to one preferable embodiment of the invention the arrangement may further comprise a sample strainer, which is arranged before the sensor in the flow direction of the sample flow during the normal operation mode, and which is arranged to effectively break up solid and particulate material, such as rags, paper debris and the like, in the sample flow before the flow comes into a contact with the sensor surface. The sample strainer may comprise a tubular outer casing and a plurality of tubular conduits arranged within the outer casing, and it has an inlet and an outlet. The tubular conduits are arranged parallel with each other and with the longitudinal axis of the outer casing between the inlet and the outlet. The outer casing and the tubular conduits usually have an identical length. The outer casing has a first diameter, for example 75-100 mm, and the tubular conduits have a second diameter, which is smaller than the first diameter, for example 5-10 mm. The exact size of the first and second diameters depend on the application. The tubular conduits usually have all identical second diameter, but in some embodiments the second diameter of the individual tubular conduits may be different from each other. The number of tubular conduits within the outer casing may be 10-30, preferably 15-25. The outer casing and the tubular conduits may be made of any suitable material, for example polymer, such as polyvinyl chloride, or metal, such as stainless steel. The length of the sample strainer may be 150-350 mm, preferably 200-300 mm.

The spaces between the tubular conduits and the outer casing in the sample strainer may be filled with any suitable filling material or resin, e.g. epoxy resin. In this manner the sample flow is forced under pressure against and through the tubular conduits. At the inlet solid and particulate material is pressed against the ends of the tubular conduits and broken apart into smaller pieces, which are pushed through the tubular conduits to the outlet. The inlet and the outlet of the sample strainer have fittings, which enable its connection with the other parts of the arrangement. The sample strainer may be used instead of or in addition to the filter. The sample strainer may also be used in other applications.

In order to prevent the plugging of the filter and/or the sample strainer, the cleaning liquid flow is arranged to flow towards and through the filter and/or the sample strainer during the cleaning cycle for backflushing the filter.

According to one embodiment of the invention the arrangement comprises means for feeding pressurized gas to the cleaning liquid flow. Pressurized gas, preferably pressurized air, may be fed to the cleaning liquid flow in order to increase the velocity of the cleaning liquid and to enhance the cleaning effect. The pressurized gas creates turbulent flow conditions especially in the vicinity of and at the sensor surface, which even more effectively remove attached dirt and impurities from the sensor surface.

All the parts of the arrangement may be arranged inside a single box-like unit. The box-like unit may comprise outer walls, made e.g. from metal, which protect the various parts of the arrangement.

### BRIEF DESCRIPTION OF THE DRAWINGS

FIG. 1 illustrates an arrangement and method according to one embodiment of the invention.

### DETAILED DESCRIPTION OF THE INVENTION

Some embodiments of the invention are explained more closely in the schematical non-limiting drawings.

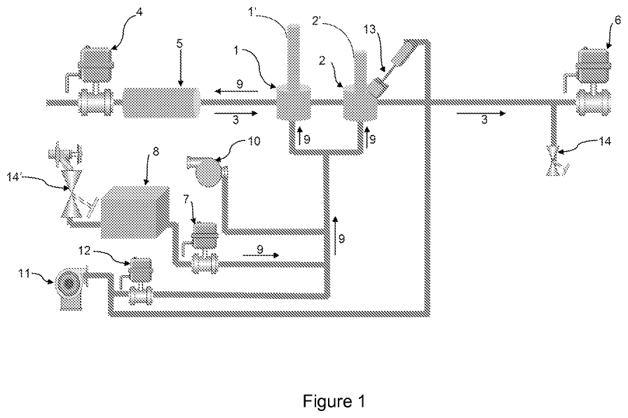

The monitoring arrangement illustrated in FIG. 1 comprises two sensors 1, 2 arranged after each other in a sample flow of wastewater, which flow direction during normal operation is denoted with arrows 3. The first sensor 1 is a pH measuring sensor and the second sensor 2 is a sensor measuring sulphide content. During normal operation mode the sensor probes 1′, 2′ are arranged in contact with the sample flow which flows past their sensor surfaces (not shown). The sample flow may be taken as a small by-pass flow from a main flow of receiving waters. The flow is led through an actuated sample inlet valve 4 and filtered by using a filter 5 arranged before the sensors 1, 2. The filter 5 removes possible solid and particulate material from the wastewater before it is led to sensors, thus reducing the exposure of the sensor surfaces to abrasive material and minimising the risk for flow connection blockage of the arrangement. Alternatively, or in addition to filter 5, a sample strainer may be arranged before the sensors. After the passing the sensors 1, 2 the sample flow 3 is discharged through an actuated sample discharge valve 6. During the normal operation the sample inlet valve 4 and sample outlet valve are open and enable the wastewater sample flow past the sensors 1, 2.

When a cleaning cycle starts the sample discharge valve 6 is closed and the sample flow 3 is effectively discontinued. An actuated cleaning liquid valve 7 is opened and a flow of cleaning liquid, e.g. water, is started from a first reservoir 8 for a cleaning liquid. The first reservoir 8 may comprise means for heating the cleaning liquid. It has been seen that the use of heated cleaning liquid may significantly improve the cleaning results obtained. The cleaning liquid is led from the first reservoir 8 towards the sensor surfaces of the sensors 1, 2, where it effectively removes dirt, such as fat and grease attached to the sensor surfaces. The flow direction of the cleaning liquid is denoted with arrows 9.

After passing the sensor surfaces the cleaning liquid flow is arranged to flow towards and through the filter 5. In this manner the cleaning liquid flow effectively also backflushes the filter 5 and improves its functioning and lifetime.

It is possible to use also a chemical cleaning agent for cleaning the sensor surfaces. Chemical cleaning agent may be introduced into the cleaning liquid flow from a second reservoir (not shown) by using a second transfer means 10 for chemical cleaning agent. The desired chemical cleaning agent amount may be easily adjusted by adjusting the volume pumped by the second transfer means 10. The chemical cleaning agent is fed into the cleaning liquid flow and mixed with the cleaning liquid during the transfer to the sensors 1, 2.

In order to maximize the cleaning result pressurized gas, e.g. compressed air, is fed to the cleaning liquid flow. The arrangement comprises means 11 for providing pressurized gas into cleaning liquid flow by opening an actuated gas valve 12. When pressurized gas is fed to the cleaning liquid flow, it causes turbulent flow conditions at the sensor surfaces, which even more effectively remove attached dirt and impurities from the sensor surface.

The sensor surface is further mechanically cleaned by using an automatic cleaning device 13. In FIG. 1 the automatic cleaning device 13 is schematically represented, but in practice it may be a wiper, brush or the like, which is brought into contact with the sensor surface, and used to detach and/or remove dirt and/or impurities from the sensor surface.

During the mechanical cleaning the valves 4, 6, 7, 12 may be closed, i.e. no flows are occurring in the arrangement during mechanical cleaning.

After mechanical cleaning the sensor surface may be rinsed by opening the cleaning liquid valve 7 and the sample discharge valve 6 and allowing cleaning liquid to flow pass the sensors 1, 2 and out through the sample discharge. After sufficient rinsing the normal operation mode of the monitoring is resumed.

The arrangement may comprise a required number of pressure relief valves for preventing pressure build-up within the arrangement.

Even if the invention was described with reference to what at present seems to be the most practical and preferred embodiments, it is appreciated that the invention shall not be limited to the embodiments described above, but the invention is intended to cover also different modifications and equivalent technical solutions within the scope of the enclosed claims.

1. A method for cleaning a sensor in a wastewater monitoring arrangement comprising at least one sensor with a sensor surface, such as an optical sensor with a window, lens, or the like, in which monitoring arrangement, during a normal operation mode, a sample flow of wastewater is arranged to flow past the sensor surface and the sensor is arranged to provide measurement values that describe a quality parameter of the wastewater, the method comprising steps of:
starting a sensor cleaning cycle by discontinuing the sample flow;
starting a cleaning liquid flow;
arranging the cleaning liquid flow towards the sensor surface;
mechanically cleaning the sensor surface by an automatic cleaning device;
discontinuing the cleaning liquid flow after a predetermined cleaning time and ending the cleaning cycle; and
starting the wastewater flow again.

2. The method according to claim 1, wherein the cleaning liquid flow comprises at least one chemical cleaning agent, such as a detergent, at least during a part of the cleaning cycle.

3. The method according to claim 1, wherein pressurized gas, preferably pressurized air, is fed to the cleaning liquid flow.

4. The method according to claim 1, wherein the cleaning liquid has an elevated temperature of at least 50° C., preferably at least 70° C., more preferably at least 75° C.

5. The method according to claim 1, wherein a filter is arranged before the sensor, in a flow direction of the sample flow during the normal operation mode, for removal solid and particulate material from the sample flow.

6. The method according to claim 1, wherein a sample strainer is arranged before the sensor, in a flow direction of the sample flow during the normal operation mode, to break up solid and particulate material, such as rags, paper debris and the like, in the sample flow before the flow comes into a contact with the sensor surface.

7. The method according to claim 5, wherein during the cleaning cycle the cleaning liquid flow is arranged to flow towards the filter for backflushing the filter.

8. The method according to claim 1, wherein the cleaning liquid flow is arranged towards the sensor surface at a straight angle.

9. The method according to claim 1, wherein the cleaning cycle is activated at predetermined time intervals.

10. A method to monitor wastewater in municipal wastewater treatment; food industry; agriculture; livestock farming; paper, board or pulp industry; or metallurgical industry, the method comprising cleaning a sensor in a wastewater monitoring arrangement according to claim 1.

11. An arrangement for a wastewater monitoring, comprising at least one sensor with a sensor surface, such as an optical sensor with a window, lens, or the like, in which arrangement during a normal operation mode a sample flow of wastewater is arranged to flow past the sensor surface and the sensor is arranged to provide measurement values which describe a quality parameter of the wastewater, the arrangement comprising
a first reservoir for a cleaning liquid, connections for leading the cleaning liquid from the first reservoir towards the sensor surface, and transfer means, such as a cleaning liquid pump, for transferring the cleaning liquid from the first reservoir to the sensor,
optional means for heating the cleaning liquid to an elevated temperature;
automatic cleaning device for mechanically cleaning the sensor surface;
a control unit, which is in functional contact with at least the transfer means and the automatic cleaning device, and which is arranged to carry out a cleaning cycle according to claim 1.

12. The arrangement according to claim 11, wherein the arrangement comprises a second reservoir for a chemical cleaning agent, connections for leading the chemical cleaning agent to the sensor, and a second transfer means, such as pump, for transferring the chemical cleaning agent.

13. The arrangement according to claim 11, wherein the arrangement comprises means for feeding pressurized gas to the cleaning liquid flow.

14. The arrangement according to claim 11, wherein the arrangement comprises a filter, which is arranged before the sensor in a flow direction of the sample flow during the normal operation mode, for removal solid and particulate material from the sample flow.

15. The arrangement according to claim 11, wherein the arrangement comprises a sample strainer, which is arranged before the sensor in the flow direction of the sample flow during the normal operation mode, and which is arranged to break up solid and particulate material, such as rags, paper debris and the like, in the sample flow.

16. The arrangement according to claim 11, wherein the arrangement comprises at least one sensor providing direct or indirect measurement data on turbidity, conductivity, pH, chemical oxygen demand (COD), biological oxygen demand (BOD), total dissolved gas (TDG), content of specific heavy metals, content of various species of nitrogen, sulfur and/or phosphorous species.

17. The arrangement according to claim 11, wherein all parts of the arrangement are arranged inside a single box-like unit.

18. The method according to claim 6, wherein during the cleaning cycle the cleaning liquid flow is arranged to flow towards the sample strainer for backflushing the filter the sample strainer.

19. The method of claim 1, wherein the wastewater is municipal wastewater; food industry wastewater; agriculture wastewater; livestock farming wastewater; wastewater from paper, board or pulp mill; or wastewater from metallurgical industry.
